(19) United States
(12) Patent Application Publication
(10) Pub. No.: US 2024/0384237 A1
(43) Pub. Date: Nov. 21, 2024

(54) DOUBLE-LAYER STRUCTURE CAPABLE OF FORMING WRINKLE STRUCTURE BY COMPRESSIVE FORCE AND METHOD FOR MANUFACTURING SAME

(71) Applicant: POSTECH RESEARCH AND BUSINESS DEVELOPMENT FOUNDATION, Pohang-si (KR)

(72) Inventors: Dong Sung KIM, Pohang-si (KR); Jae Seung YOUN, Pohang-si (KR)

(21) Appl. No.: 18/641,674

(22) Filed: Apr. 22, 2024

(30) Foreign Application Priority Data
Apr. 26, 2023 (KR) 10-2023-0054444

(51) Int. Cl. C12N 5/071; C08J 3/075

(52) U.S. Cl. CPC C12N 5/0679; C08J 3/075; C12N 5/0625

(57) ABSTRACT

The present disclosure relates to a double-layer structure including: a first layer composed of a gel composition; a second layer formed on the first layer and having an epithelial cell layer as at least a region coming into contact with the first layer; and a wrinkle structure formed thereon by means of compressive forces applied from the outside thereto, the wrinkle structure having at least one or more crests or troughs formed thereon in a state where the first layer and the second layer are connected to each other.

### BACKGROUND OF THE DISCLOSURE

### Cross Reference to Related Application of the Disclosure

The present application claims the benefit of Korean Patent Application No.10-2023-0054444 filed in the Korean Intellectual Property Office on Apr. 26, 2023, the entire contents of which are incorporated herein by reference.

### Field of the Disclosure

The present disclosure relates to a double-layer structure capable of forming a solid wrinkle structure by means of a compressive force and a method for manufacturing the same, more specifically to a double-layer structure that is provided with a first layer composed of a gel composition and a second layer formed on the first layer and having an epithelial cell layer, thereby capable of forming a wrinkle or fold structure thereon when a compressive force is applied thereto and to a method for manufacturing the same.

### Background of the Related Art

Epithelial tissues of the skin and intestine of a human body naturally have wrinkle or fold structures by means of forces (compressive forces) applied from the outside. The wrinkle or fold structures of the epithelial tissues are important structures that are necessarily considered in studies of embryology and pathology, and only if such natural wrinkle or fold structures of the epithelial tissues are artificially made, improvements in cosmetic products as well as developments of academic studies can be obtained.

A conventional material structure made by copying the epithelial tissue is proposed, but such a conventional structure has limitations in providing the wrinkle or fold structure similar to the wrinkle or fold structure the real epithelial tissue has. This is because the conventional structure does not have materials capable of copying a layered structure of the epithelial tissue and fails to provide a layered structure similar to the real layered structure.

The present disclosure is proposed to solve such problems and relates to a double-layer structure having the similar characteristics to the real epithelial tissue and a method for manufacturing the double-layer structure. Accordingly, the present disclosure removes the technical problems as mentioned above and provides additional technical elements that are not easily invented by a person having ordinary skill in the art.

Prior Art Document

Patent Document

Korean Patent Application Laid-open No. 10-2020-0040968 (Dated on Apr. 21, 2020)

### SUMMARY OF THE DISCLOSURE

Accordingly, the present disclosure has been made in view of the above-mentioned problems occurring in the related art, and it is an object of the present disclosure to provide a double-layer structure capable of naturally forming a wrinkle or fold structure when external forces are applied thereto and a method for manufacturing the same. In specific, explanations of compositions or environments through which components of the double-layer structure are made and the characteristics of the double-layer structure appearing when compressive forces are applied under such compositions or environments will be given to help the double-layer structure and the method for manufacturing the same understood.

It is another object of the present disclosure to provide a device that is capable of compressing a double-layer structure, that is, a device that is provided with components adequate for the structural characteristics of the double-layer structure and for compression test environments of the double-layer structure, thereby allowing the double-layer structure to be smoothly compressed and observed.

To accomplish the above-mentioned objects, according to one aspect of the present disclosure, there is provided a double-layer structure including: a first layer composed of a gel composition; a second layer formed on the first layer and having an epithelial cell layer as at least a region coming into contact with the first layer; and a wrinkle structure formed thereon by means of compressive forces applied from the outside thereto, the wrinkle structure having at least one or more crests or troughs formed thereon in a state where the first layer and the second layer are connected to each other.

According to the present disclosure, desirably, the wrinkle structure may be formed when a compressive strain of the double-layer structure is in the range of 0.2 to 0.7.

To accomplish the above-mentioned objects, according to another aspect of the present disclosure, there is provided a double-layer structure including: a first layer composed of a gel composition; a second layer formed on the first layer and having an epithelial cell layer as at least a region coming into contact with the first layer; and a fold structure formed thereon by means of compressive forces applied from the outside thereto, the fold structure having a V-shaped folded region formed thereon in a state where the first layer and the second layer are connected to each other.

According to the present disclosure, desirably, the fold structure may be formed when a compressive strain of the double-layer structure is greater than or equal to 0.7.

To accomplish the above-mentioned objects, according to yet another aspect of the present disclosure, there is provided a method for manufacturing a double-layer structure, including the steps of: forming a first layer composed of a gel composition and forming a second layer on top of the first layer; and applying compressive forces to the outside of the first layer or the second layer by means of at least one or more pressurizing parts to dehydrate the first layer, wherein at least a region of the second layer is an epithelial cell layer coming into contact with the first layer.

According to the present disclosure, desirably, at the step of applying the compressive forces, each pressurizing part may have a compression rate of 0.001 to 0.5 mm/s.

To accomplish the above-mentioned objects, according to still another aspect of the present disclosure, there is provided a device for compressing a double-layer structure having a first layer composed of a gel composition and a second layer formed on the first layer and having an epithelial cell layer as at least a region coming into contact with the first layer, the device including: a base for putting the double-layer structure on top thereof; a fixing part located on top of the base to fix the double-layer structure thereto; and a pressurizing part for applying a compressive force to the double-layer structure.

According to the present disclosure, desirably, the device may further include an observing part for observing the double-layer structure.

### BRIEF DESCRIPTION OF THE DRAWINGS

The above and other objects, features and advantages of the present disclosure will be apparent from the following detailed description of the embodiments of the disclosure in conjunction with the accompanying drawings, in which:

FIGS. 1A and 1B show a double-layer structure according to the present disclosure, especially before compressive forces are applied thereto and after the compressive forces have been applied thereto;

FIG. 2 is a flowchart showing a method for manufacturing the double-layer structure according to the present disclosure;

FIG. 3 is a graph showing changes in aspect ratios of the double-layer structure when the compressive forces are applied to the double-layer structure;

FIG. 4 is a graph showing a process of forming a wrinkle or fold structure on the double-layer structure according to compressive strains;

FIG. 5 shows the fold structure formed on the double-layer structure according to the present disclosure;

FIGS. 6A to 6D show graphs showing the wrinkle-to-fold transition of the double-layer structure according to the present disclosure;

FIGS. 7A and 7B show the double-layer structure really manufactured according to the present disclosure;

FIG. 8 is a graph showing states where the wrinkle structures are formed according to compression rates;

FIG. 9 shows states where the deformation of the double-layer structure according to hydrogel concentrations;

FIGS. 10A, 10B, 11A, 11B and 11C show the wrinkle structures that are formed when the compressive forces are applied thereto in various ways;

FIG. 12 is a side view showing a compression device for forming the wrinkle or fold structure on the double-layer structure according to the present disclosure; and

FIG. 13 is a photograph showing an example of the double-layer structure really compressed by means of the compression device.

### DETAILED DESCRIPTION OF THE PREFERRED EMBODIMENT

Hereinafter, an explanation of the present disclosure will be given in detail with reference to the attached drawings. Objects, characteristics and advantages of the present disclosure will be more clearly understood from the detailed description as will be described below and the attached drawings. Before the present disclosure is disclosed and described, it is to be understood that the disclosed embodiments are merely exemplary of the disclosure, which can be embodied in various forms. Therefore, specific structural and functional details disclosed herein are not to be interpreted as limiting, but merely as a basis for the claims and as a representative basis for teaching one of ordinary skill in the art to variously employ the present disclosure in virtually any appropriately detailed structure. In the description, the same reference numerals will be used to describe the same components.

All terms used herein, including technical or scientific terms, unless otherwise defined, have the same meanings which are typically understood by those having ordinary skill in the art. The terms, such as ones defined in common dictionaries, should be interpreted as having the same meanings as terms in the context of pertinent technology, and should not be interpreted as having ideal or excessively formal meanings unless clearly defined in the specification. Terms used in this application are used to only describe specific exemplary embodiments and are not intended to restrict the present disclosure. An expression referencing a singular value additionally refers to a corresponding expression of the plural number, unless explicitly limited otherwise by the context.

Terms, such as the first, the second, and the like may be used to describe various elements, but the elements should not be restricted by the terms. The terms are used to only distinguish one element from the other element. For example, a first element may be named a second element without departing from the scope of the present disclosure. Likewise, a second element may be named a first element.

In this application, terms, such as “comprise”, “include”, or ‘have ”, are intended to designate those characteristics, numbers, steps, operations, elements, or parts which are described in the specification, or any combination of them that exist, and it should be understood that they do not preclude the possibility of the existence or possible addition of one or more additional characteristics, numbers, steps, operations, elements, or parts, or combinations thereof.

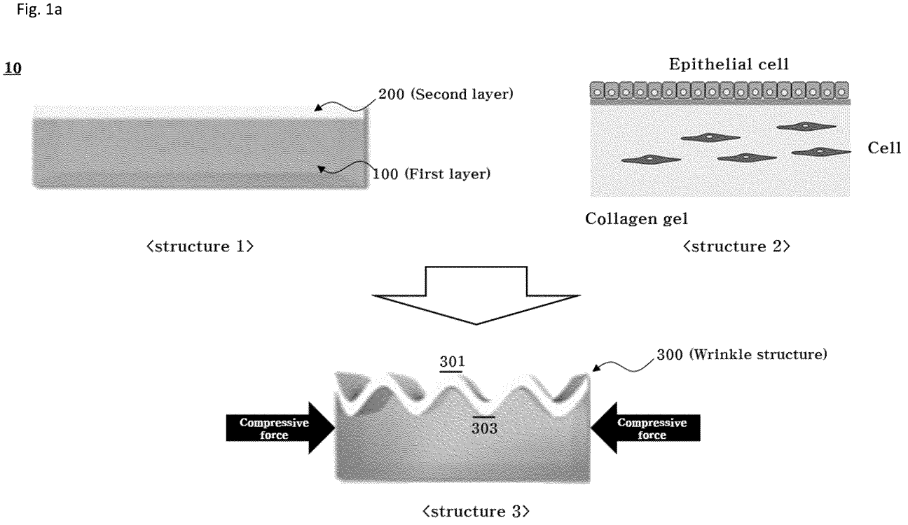

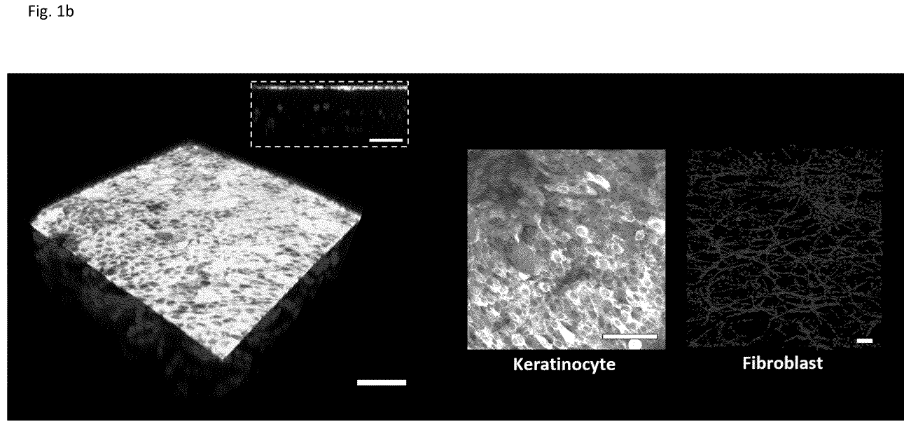

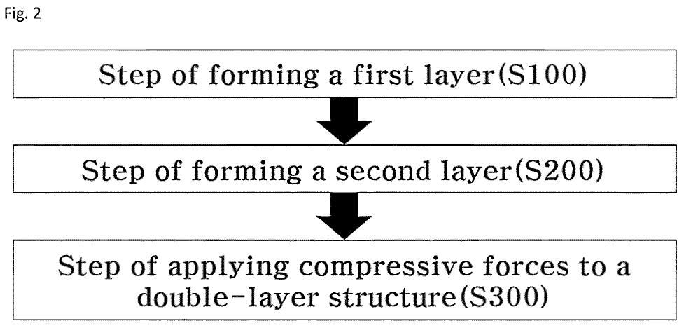

A structure 1 as shown in FIG. 1A shows a double-layer structure 10 according to the present disclosure, and FIG. 2 shows a method for manufacturing the double-layer structure 10 according to the present disclosure. As shown, the double-layer structure 10 includes a first layer 100 located on the bottom thereof and a second layer 200 located on top of the first layer 100. Referring to FIG. 2, the method for manufacturing the double-layer structure 10 includes the steps of forming the first layer 100 (at step S100), forming the second layer 200 on top of the first layer 100 (at step S200), and applying compressive forces to the double-layer structure 10 to form a wrinkle or fold structure on the double-layer structure 10. Hereinafter, explanations of the respectively layers of the double-layer structure 10 and the method for forming such layers will be given.

The first layer 100 is composed of a gel composition having viscoelasticity and basically serves as a support body. Desirably, the first layer 100 is composed of a hydrogel composition made by mixing a collagen solution having a concentration of 0.5 to 20 mg/ml melted in an acid solvent, an 1 M NaOH aqueous solution, and a highly concentrated PBS or cell culture medium (whose concentration has 10 times higher than a typical ion concentration) at the volume ratio of 1:0.025:0.1 to the form of a gel. The process of manufacturing the first layer 100 includes the step of neutralizing a collagen solution to a pH value of 7 by means of NaOH and simultaneously putting phosphate ions into the first layer 100. In the process of manufacturing the first layer 100, a thickness of the first layer 100 is determined according to a manufacturer's intention, and therefore, the thickness of the first layer 100 is determined according to the adjustment of a height of the gel composition for forming the first layer 100 in a container. The thickness of the first layer 100 is at least 4 times higher than a thickness of the second layer 200 as will be discussed later, and if the thickness of the first layer 100 is 4 times lower than the thickness of the second layer 200, a wrinkle or fold structure may not be formed well on the first layer 100 when compressive forces are applied to the first layer 100 from the outside. When the thickness of the first layer 100 is 10 times higher than the thickness of the second layer 200, desirably, it is checked that the wrinkle or fold structure is formed well on the first layer 100.

The second layer 200 is formed on top of the first layer 100 and includes an epithelial cell layer as at least a portion coming into contact with the first layer 100. According to the present disclosure, desirably, the second layer 200 is composed of only the epithelial cell layer, and a region of the second layer 200 is greater than desirably 85%, more desirably 90% of the plane region of the first layer 100 when the first layer 100 is viewed on top thereof. The second layer 200 is made by cultivating cells on the first layer 100, and for example, cells having a concentration of 1×10⁵ to 5×10⁶ cells/ml are inoculated and then cultivated for 24 hours, thereby forming the second layer 200 having the epithelial cell layer.

The first layer 100 and the second layer 200 are essential components of the double-layer structure 10, and in specific, the first layer 100 and the second layer 200 are not formed as the double layer by means of simple lamination and adhesion. Accordingly, they have an organic connection structure therebetween. In this case, proteins or protein complexes constituting the organic connection structure between the first layer 100 and the second layer 200 include integrins, hemidesmosomes, focal adhesions, etc.

The structure 1 as shown in FIG. 1A shows the double-layer structure 10 conceptually divided into the first layer 100 and the second layer 200, and a structure 2 as shown in FIG. 1B shows a state wherein a collagen gel as the first layer 100 and the epithelial cell layer as the second layer 200 are laminated on top of each other. According to the present disclosure, in this case, different types of cells from the epithelial cell layer are contained in the collagen gel, that is, the first layer 100, and for example, immune cells or fibroblasts are contained in the first layer 100. The double-layer structure 10 of the present disclosure is made to copy the epithelial tissue (epithelial layer-connective tissue layer) of the human body, and like the structure 2 as shown in FIG. 1B, if the immune cells or fibroblasts are contained in the first layer 100, an environment more similar to the epithelial tissue of the human body is made. The wrinkle or fold structure formed in the double-layer structure 10 is influenced by the composition of the first layer 100, and according to the present disclosure, the environment more similar to the epithelial tissue of the human body is made so that the deformation of the wrinkle or fold structure may be more accurately understood.

To allow the cells to be additionally contained in the first layer 100, that is, the collagen gel, further, the cells to be contained are mixed in a process of making the collagen gel, that is, in a process of mixing the collagen solution, NaOH, and PBS (or cell culture medium), and then made to the form of gel. In this case, a mixing concentration of the cells to be contained is in the range of 1×10⁴ to 1×10⁷ cells/ml.

According to the present disclosure, in the structure 1 as shown in FIG. 1A or in the structure 2 as shown in FIG. 1B, the second layer or epithelial cell layer is any one of the intestine epithelial cell layer, the lung epithelial cell layer, the kidney epithelial cell layer, and the skin epithelial cell layer.

Further, adherens junctions exist in the epithelial cells formed as the second layer 200 to perform epithelial cell-epithelial cell connection, and such adherens junctions are composed of epithelial cadherins. Furthermore, peripheral actin rings may exist in the epithelial cells.

In specific, FIG. 1B shows a state wherein the double-layer structure 10 consists of the first layer on which fibroblasts are contained in the collagen gel and the second layer as a layer of keratinocytes formed on top of the first layer. A portion inside a dotted line as shown in FIG. 1B shows the double-layer structure 10 observed on the side thereof, and in this case, it is checked that a relatively thin layer of keratinocytes is formed on top of the collagen gel having a relatively high thickness.

A structure 3 as shown in FIG. 1A shows a state wherein a wrinkle structure occurs when compressive forces are applied to the double-layer structure 10 as the structure 1 or 2. Referring to the structure 3 as shown in FIG. 1A and the step S300 of FIG. 2, if external forces are applied to the double-layer structure 10, a wrinkle structure 300 is made on the double-layer structure 10, and in this case, the wrinkle structure 300 has at least one or more crests 301 or/and troughs 303 in a state where the first layer 100 and the second layer 200 are connected to each other. In more specific, the wrinkle structure 300 has crests 301 or/and troughs 303 repeatedly formed thereon. In the description, the external forces are called compressive forces for convenience.

The compressive forces are not limited in the directions of the forces applied, the strength of the forces, and the like only if they induce the double-layer structure 10 to be structurally deformed. Further, it can be appreciated that strength points to which the compressive forces are applied are freely determined. When the hexahedron-shaped double-layer structure 10 as shown in the Figures is viewed, the compressive forces are applied to both left and right sides of the double-layer structure 10. In specific, the points to which the compressive forces are applied are arbitrary two points of the outside of the first layer 100, and the two points are set to be located at the same height.

As the compressive forces are applied to the double-layer structure 10 to cause the double-layer structure 10 to be deformed, the wrinkle structure 300 is made, and according to the present disclosure, an explanation of conditions for making the wrinkle structure 300 using a parameter such as a compressive strain will be given.

The compressive strain is the change in a linear distance between both ends of a structure due to a compressive force acting on the structure. In detail, the compressive strain is determined by dividing the linear distance value between both ends of the double-layer structure 10 deformed due to the compressive forces applied thereto by the linear distance value between both ends of the double-layer structure 10 to which the compressive forces are not applied. For example, if it is assumed that the linear distance value between both ends of the double-layer structure 10 to which the compressive forces are not applied is 20 cm and the linear distance value between both ends of the double-layer structure 10 deformed due to the compressive forces applied thereto is 12 cm, the linear distance value is reduced by 8 cm by means of the compressive forces, and accordingly, the compressive strain is 0.4 or 40%. According to the present disclosure, the deformation conditions of the double-layer structure 10 are explained, based on the compressive strain as a parameter, and further, it can be appreciated that the compressive strain is proportional to the magnitudes of the compressive forces applied to the double-layer structure 10. As shown in FIG. 1A, the compressive strain is a measurable parameter when the compressive forces (In this case, the magnitude values of the compressive forces are considered) are applied to the sides of the double-layer structure 10, and as the magnitude values of the compressive forces become higher, in this case, it can be appreciated that the compressive strains increase up to a given level in proportion to the compressive forces.

Referring back to the conditions for making the wrinkle structure 300, the wrinkle structure 300 of the double-layer structure 10 is made as follows. First, the first layer 100 having the characteristics of gel (hydrogel) is compressed to a relatively high compressive strain, and a compressive strain of the second layer 200 having the epithelial cell layer is less than 0.4, so that a relatively big difference between the compressive strains is made when the compressive forces are applied to the first layer 100 and the second layer 200, thereby making the wrinkle structure 300. That is, if the compressive forces are applied to the double-layer structure 10 having the first layer 100 and the second layer 200 so that the compressive strain of the double-layer structure 10 is greater than or equal to 0.2, desirably greater than or equal to 0.4, the second layer 200 having the epithelial cell layer is not compressed anymore, thereby making the wrinkle structure 300.

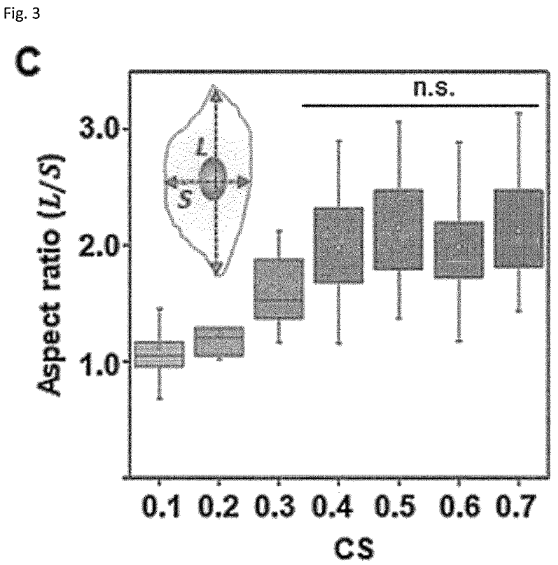

FIG. 3 shows a graph wherein rare changes are made in aspect ratios of single epithelial cells constituting the double-layer structure 10 in the case where the compressive forces are applied to the double-layer structure 10 to cause the compressive strain of the double-layer structure 10 to be greater than or equal to 0.4.

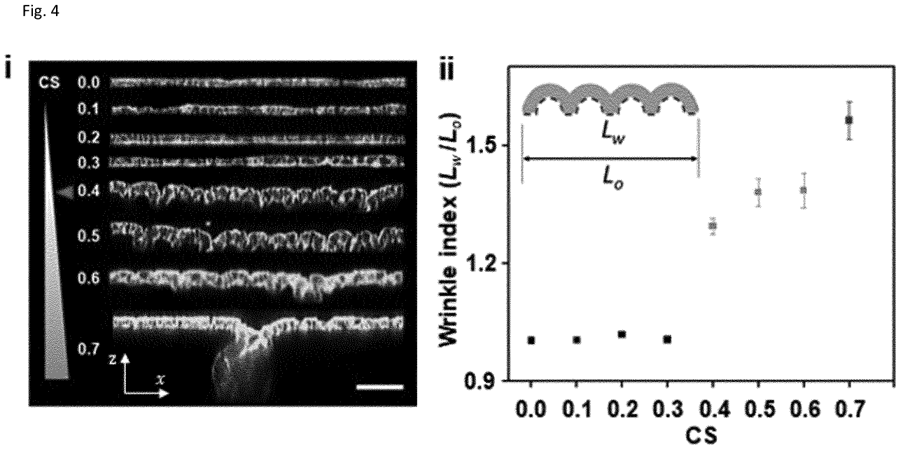

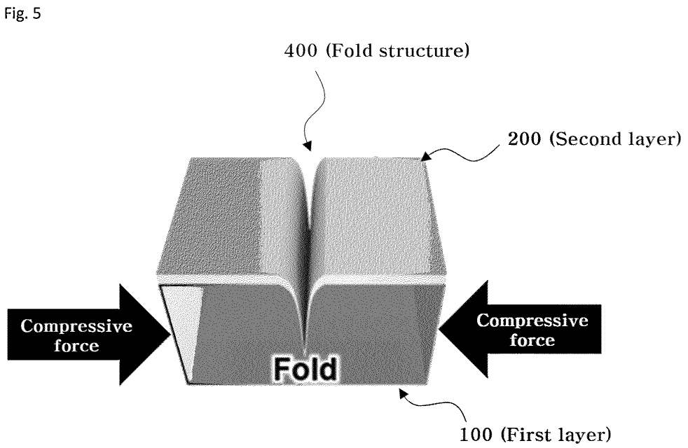

FIG. 4 shows a state where the wrinkle structure 300 starts to be made when the compressive forces are applied to the double-layer structure 10 to cause the compressive strain of the double-layer structure 10 to be greater than or equal to 0.4 and a state where a fold structure 400 (See FIG. 5), not the wrinkle structure 300, is made when the compressive forces are applied to the double-layer structure 10 in such a way as to induce a compressive strain greater than or equal to 0.7. That is, a threshold value of the compressive strain for making the wrinkle structure 300 is greater than or equal to 0.2 and less than 0.7, desirably greater than or equal to 0.4 and less than 0.7, and a threshold value of the compressive strain for making the fold structure 400 is greater than or equal to 0.7.

FIG. 5 shows the fold structure 400, and when the compressive forces are applied to the double-layer structure 10 in such a way as to cause the compressive strain greater than or equal to the threshold value, the fold structure 400, not the wrinkle structure 300, is formed. In detail, the fold structure 400 is made as follows. In a state where the first layer 100 and the second layer 200 are organically connected to each other, if great compressive forces are applied to the double-layer structure 10, planes where the first layer 100 and the second layer 200 are flat exist on regions from both ends to which the compressive forces are applied to the center of the double-layer structure 10, and a “V”-shaped fold is formed on a region from both ends of the flat surfaces to a center point of the double-layer structure 10, thereby making the fold structure 400. The “V”-shaped fold may have the shape of accurate “V” or the shape similar to it. The fold structure 400 is different from the wrinkle structure 300 in that it does not have any crests or troughs repeatedly formed thereon.

In the process of applying the compressive forces to the double-layer structure 10, further, the wrinkle structure 300 and the fold structure 400 are made sequentially. That is, if the compressive forces applied to the double-layer structure 10 slowly increase, the double-layer structure 10 having the first layer 100 and the second layer 200 starts to be compressed, while maintaining the flat shape, until the compressive strain of the double-layer structure 10 reaches 0.4. The wrinkle structure 300 is formed on the double-layer structure 10 when the compressive strain of the double-layer structure 10 is greater than or equal to 0.4, and if the compressive strain is greater than 0.7, the fold structure 400 is formed on the double-layer structure 10. That is, the double-layer structure 10 is deformed sequentially.

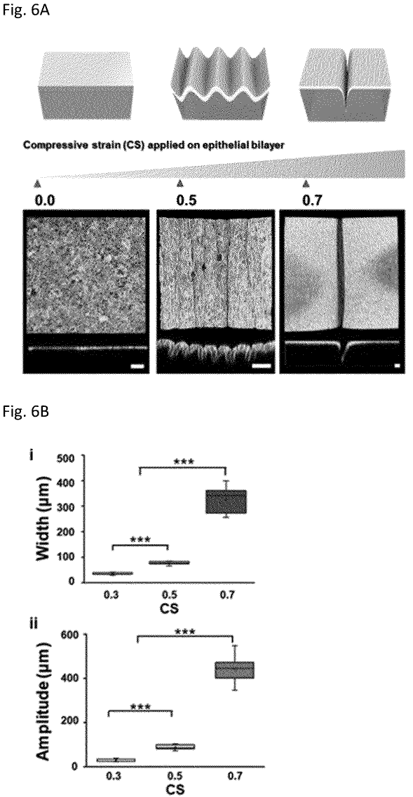

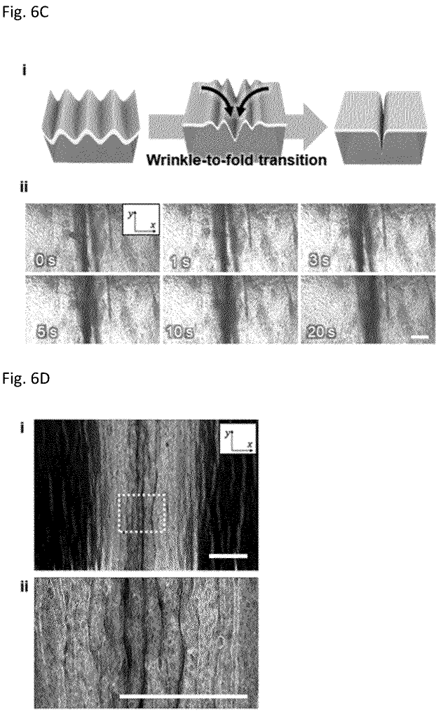

FIGS. 6A to 6D show graphs from which transition of such structures is checked. FIG. 6A shows the graph wherein the deformation of the structures formed on the double-layer structure 10 according to the increase of the compressive strain, FIG. 6B shows the graph wherein the changes in widths and amplitude values of the structures according to the increase of the compressive strain, and FIGS. 6C and 6D show a state where the uniform wrinkle structure 300 becomes irregular upon wrinkle-to-fold transition. In detail, a deeper trough is formed on a center point, and relatively low troughs are formed on the region distant from the center point. During the transition, it is observed that some wrinkles having the relatively low troughs merged to a single big wrinkle. Referring to FIG. 6B, under the compressive strain of 0.3 to 0.5, a distance between the crests is in the range of about 20 to 100 μm, and a height difference between the crest and the trough is in the range of 10 to 100 μm. When the compressive strain is 0.7, further, an open width of the fold structure 400 is measured in the range of about 250 to 350 μm, and a height (depth) of the fold structure 400 is in the range of about 400 to 500 μm. However, the values as shown in FIG. 6B are exemplary, and therefore, the measured values of the wrinkle structure 300 or the fold structure 400 may not be limited thereby.

Up to now, the double-layer structure 10 and the method for making the same according to the present disclosure have been explained with reference to FIGS. 1A to 6D.

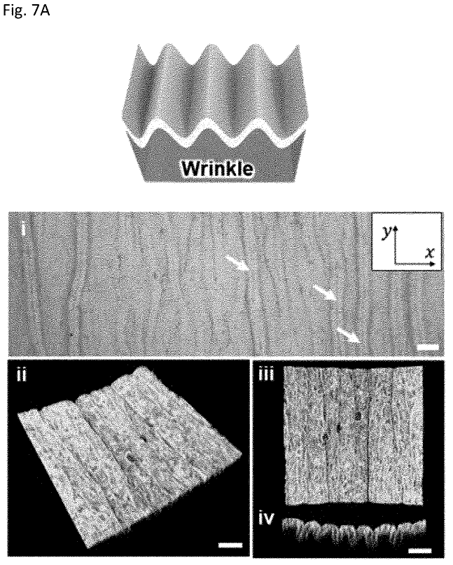

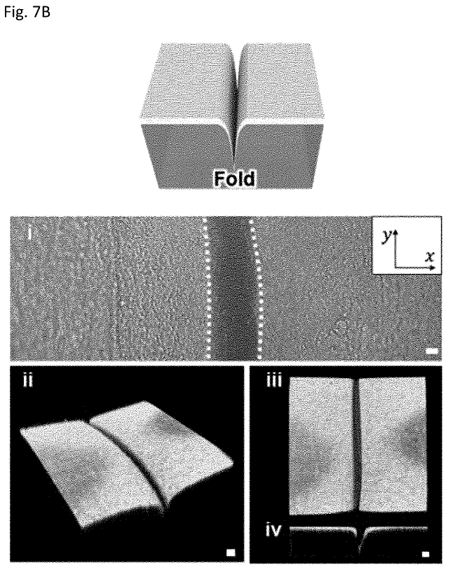

FIGS. 7A and 7B show photographs (scale bar of 100 μm) wherein the double-layer structure 10 having a collagen gel block as the first layer 100 and an epithelial cell layer as the second layer 200 is utilized as an artificial epithelial tissue. As shown, the artificial epithelial tissues having the wrinkle structure 300 and the fold structure 400 when the compressive forces are applied to the double-layer structure 10 are made to the shape as shown in FIG. 7A and the shape as shown in FIG. 7B.

When the compressive forces are applied to the double-layer structure 10, further, the deformed shapes of the structure may be differently made according to compression rates. That is, if the compressive forces are applied at fast compression rates, it is likely that the second layer 200 is entirely buckled from the first layer 100 before the wrinkle structure 300 is formed, so that the double-layer structure 10 may be damaged, thereby causing low usability. To avoid such problems from occurring, desirably, the step of applying the compressive forces is controlled to allow the compression rate to be in the range of 0.001 to 0.5 mm/s. Actually, there are many difficulties in designing the application of the compressive forces at the compression rate of 0.001 mm/s or under, and further, a lot of time for the compression becomes required to cause the survival rate of cells to seriously decrease or to fail to form regular wrinkles due to evaporation of the water in the double-layer structure. If the compression occurs at a fast compression rate exceeding 0.5 mm/s, contrarily, a wrinkle structure is not formed properly on the double-layer structure. Therefore, the compression rate is limited to the above-mentioned range. The reason why the second layer 200 is buckled under the fast compression rate is because time allowing water to be sufficiently discharged from the first layer 100 (corresponding to extracellular matrix layer) is not enough, and according to the present disclosure, the compressive forces are applied to the double-layer structure 10 at the compression rate of 0.001 to 0.5 mm/s, so that dehydration is smoothly performed to make the wrinkle structure 300 on the double-layer structure 10.

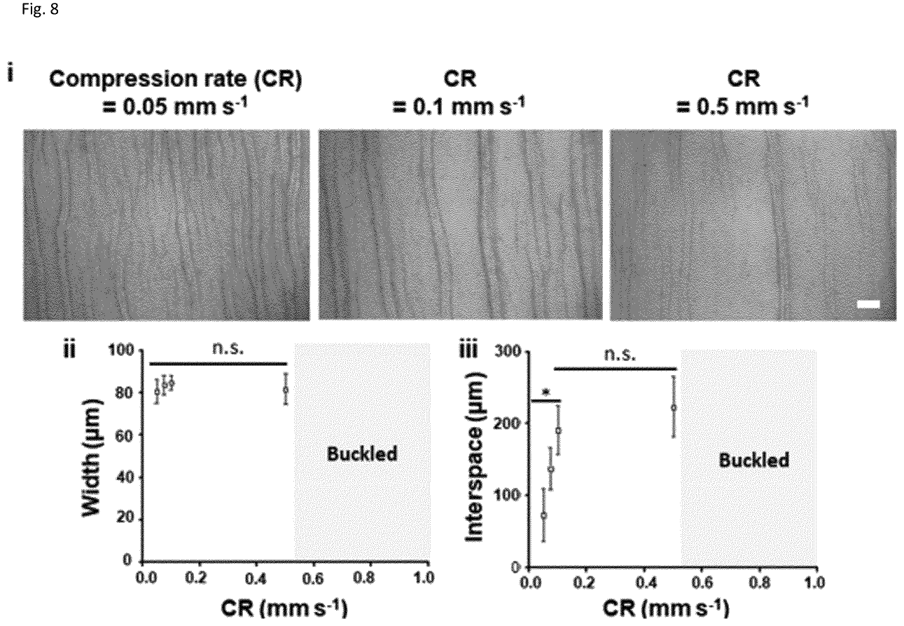

FIG. 8 shows the shapes of the wrinkle structures 300 when the compression rates are 0.05 mm/s, 0.1 mm/s, and 0.5 mm/s. When the compressive forces are applied to the double-layer structure 10 at the compression rates of 0.05 and 0.1 mm/s, relatively uniform wrinkle structures 300 are made, but when the compressive forces are applied at the compression rate of 0.5 mm/s, the regularity of the wrinkle structure 300 is drastically lowered to cause the fold structure 400 to start to be made. If the compressive forces are applied to the double-layer structure 10 at the compression rate greater than 0.5 mm/s, it can be checked that the second layer 200 is buckled, thereby making it hard to make the wrinkle structure 300 itself.

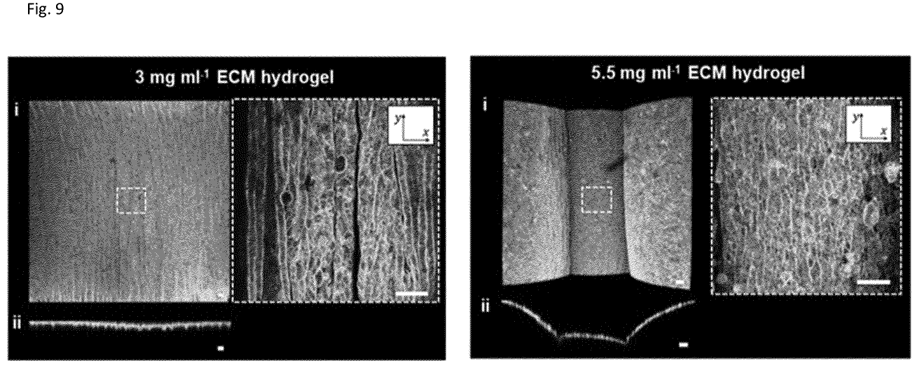

Further, the structurally deformed shape of the double-layer structure 10 may be influenced by the concentration of proteins of the first layer 100. That is, if protein concentration (e.g., type I collagen) is high to make the internal structure of the first layer 100 dense, dehydration of the first layer 100 is not smooth so that even at the same compression rate, macroscopic folds, not wrinkles, are formed. FIG. 9 shows structural deformation when 3 mg/ml ECM hydrogel and 5.5 mg/ml ECM hydrogen are used. In this case, a regular wrinkle structure 300 is made when the hydrogel with a relatively low concentration is used, and contrarily, two macroscopic fold structures are formed when the hydrogel with a relatively high concentration is used. Like this, the folds (wrinkles) are differently formed on the double-layer structure 10 according to collagen concentrations, and in consideration of the structurally deformed shapes, the protein concentration of the first layer 100 is determined. Desirably, the protein concentration of the first layer 100 is in the range of 2 to 5 mg/ml, and if the protein concentration of the first layer 100 is less than 2 mg/ml, the double-layer structure 10 fails to be kept in shape. Therefore, it is desirable that the protein concentration of the first layer 100 be greater than or equal to 2 mg/ml.

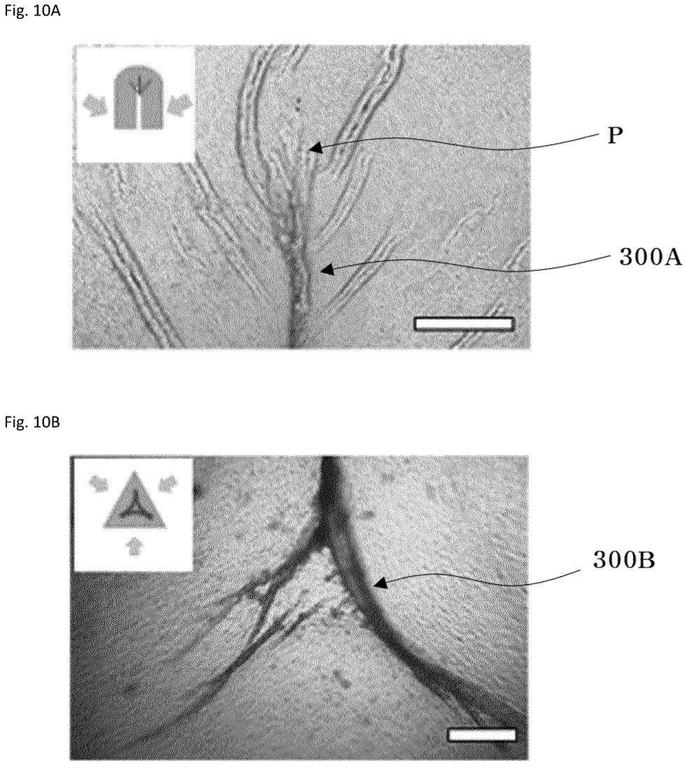

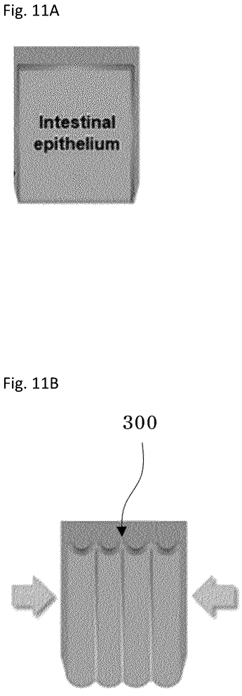

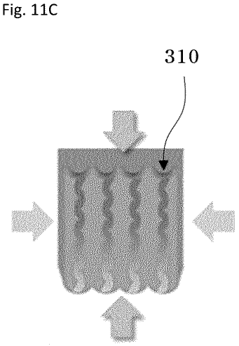

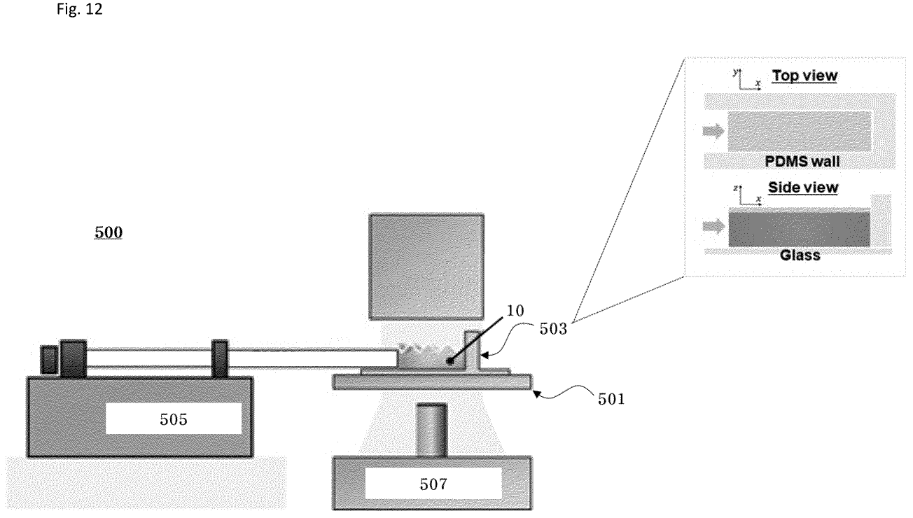

Further, it is not necessary that the application of the compressive forces to the double-layer structure 10 is made on both ends of the double-layer structure 10 as shown in FIG. 1A, and therefore, the directions of the compressive forces, the points to which the compressive forces are applied, and the number of points to which the compressive forces are applied may be not limited only if the compressive forces are applied to the double-layer structure 10 from the outside. FIGS. 10A and 10B show examples where the wrinkle structures are made when compressive forces are applied in various ways. FIG. 10A shows the example where the compressive forces are applied to bend the double-layer structure 10 having a long shape, and in this case, it can be checked that a wrinkle structure 300A is formed around a point P where the inner surfaces of the double-layer structure 10 bent are brought into contact with each other. FIG. 10B shows the example where the compressive forces are applied in three directions to allow the double-layer structure 10 to become compressed to the shape of a triangular pillar, and in this case, it can be checked that a wrinkle structure 300B is formed to the shape of a triangle. In this case, wrinkles are curved in the same directions as the direction of the compressive forces applied. FIG. 11 shows another example where the wrinkle structure is made. The compressive forces are applied to the sides of the hexahedron-shaped double-layer structure 10 to form a primary wrinkle structure 300 having a first shape (from A to B), and in the state where the primary wrinkle structure 300 is formed, compressive forces are additionally applied to top and underside of the double-layer structure 10 to form a secondary wrinkle structure 310 having a second shape. The secondary wrinkle structure 310 having the second shape has crests and troughs formed in a horizontal direction of the double-layer structure 10, unlike the primary wrinkle structure 300 having the first shape where the wrinkles are formed only in a vertical direction of the double-layer structure 10, so that the double-layer structure 10 has a relatively solid wrinkle structure. Like this, the compressive forces are applied to the double-layer structure 10 from the outside in various ways, and therefore, the shapes of the wrinkle structures may be diversely made.

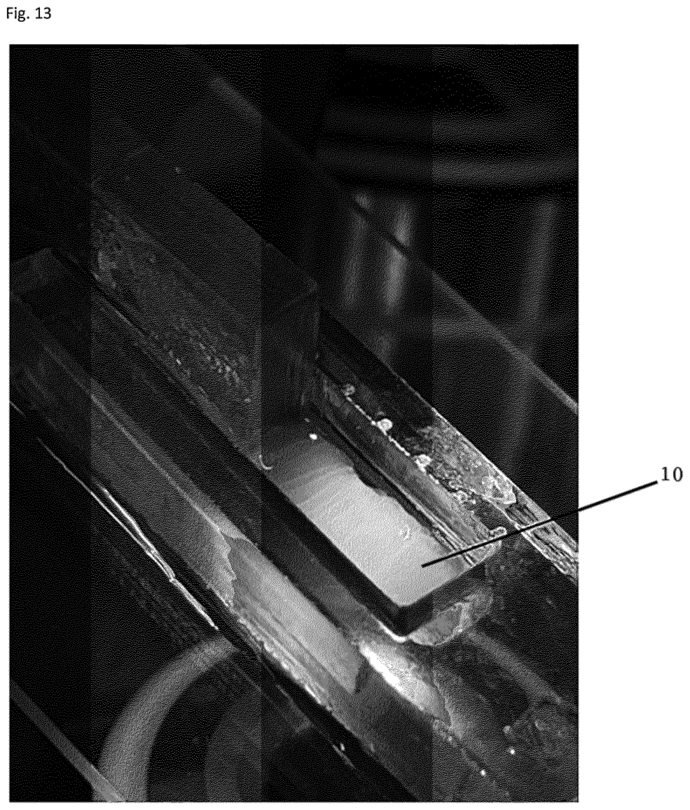

FIG. 12 shows a compression device 500 for the double-layer structure (hereinafter, referred simply to as compression device) according to another embodiment of the present disclosure, and FIG. 13 is a photograph showing an example of the double-layer structure really compressed by means of the compression device. In this case, it is checked that the double-layer structure 10 as shown in FIG. 13 has white lines appearing in a transverse direction thereof, and the white lines represent portions where wrinkles are formed by the compressive forces.

The compression device 500 includes a base 501 for putting the double-layer structure 10 on top thereof, a fixing part 503 located on top of the base 501 to fix the double-layer structure 10 thereto, a pressurizing part 505 for applying a compressive force to the double-layer structure 10, and an observing part 507 for observing the double-layer structure 10.

The base 501 serves to support the double-layer structure 10 and the fixing part 503, and therefore, only if the base 501 is made of a material capable of supporting the components, it may not be limited in material. In consideration of the position of the observing part 507 as will be discussed later, however, at least a portion of the base 501 is made of transparent glass through which the double-layer structure 10 is observed through the observing part 507.

The fixing part 503 serves to fix the double-layer structure 10 so that the double-layer structure 10 is prevented from escaping from the base 501 during the compression process thereof. The fixing part 503 may not be limited in material, and desirably, the fixing part 503 is made of an organosilicon compound such as polydimethylsiloxane (PDMS). The fixing part 503 has different shapes according to the shapes of the double-layer structure 10. Basically, the fixing part 503 has an outer wall for surroundingly fixing the outer surfaces of the double-layer structure 10 and a support wall for supporting the double-layer structure 10 so that the double-layer structure 10 is prevented from pushing when the compressive force is applied from the pressurizing part 505 to the double-layer structure 10.

As shown, the pressurizing part 505 has a rod for applying a force to the double-layer structure 10 fixed to the fixing part 503 and a motor and a substrate for controlling the operation of the rod. The pressurizing part 505 as shown is an automated part, but it is not necessary to have such automated part. That is, the pressurizing part 505 is designed to apply the compressive force to the double-layer structure 10 by means of a user's manual operation.

The observing part 507 serves to observe the double-layer structure 10 when the compressive force is applied to the double-layer structure 10 and has at least one or more lenses and a device for imaging and storing phenomena if necessary.

As described above, the double-layer structure according to the present disclosure can form the wrinkle or fold structure thereon.

Further, the double-layer structure according to the present disclosure can be compressed in various environments.

While the present disclosure has been described with reference to the particular illustrative embodiments, it is not to be restricted by the embodiments but only by the appended claims. It is to be appreciated that those skilled in the art can change or modify the embodiments without departing from the scope and spirit of the present disclosure.

What is claimed is:

1. A double-layer structure comprising:
a first layer composed of a gel composition;
a second layer formed on the first layer and having an epithelial cell layer as at least a region coming into contact with the first layer; and
a wrinkle structure formed thereon by means of compressive forces applied from the outside thereto, the wrinkle structure having at least one or more crests or troughs formed thereon in a state where the first layer and the second layer are connected to each other.

2. The double-layer structure according to claim 1, wherein the wrinkle structure is formed when a compressive strain of the double-layer structure is in the range of 0.2 to 0.7.

3. A double-layer structure comprising:
a first layer composed of a gel composition;
a second layer formed on the first layer and having an epithelial cell layer as at least a region coming into contact with the first layer; and
a fold structure formed thereon by means of compressive forces applied from the outside thereto, the fold structure having a V-shaped folded region formed thereon in a state where the first layer and the second layer are connected to each other.

4. The double-layer structure according to claim 3, wherein the fold structure is formed when a compressive strain of the double-layer structure is greater than or equal to 0.7.

5. A method for manufacturing a double-layer structure, comprising the steps of:
forming a first layer composed of a gel composition and forming a second layer on top of the first layer; and
applying compressive forces to the outside of the first layer or the second layer by means of at least one or more pressurizing parts to dehydrate the first layer,
wherein at least a region of the second layer is an epithelial cell layer coming into contact with the first layer.

6. The method according to claim 5, wherein at the step of applying the compressive forces, each pressurizing part has a compression rate of 0.001 to 0.5 mm/s.

7. A device for compressing a double-layer structure having a first layer composed of a gel composition and a second layer formed on the first layer and having an epithelial cell layer as at least a region coming into contact with the first layer, the device comprising:
a base for putting the double-layer structure on top thereof;
a fixing part located on top of the base to fix the double-layer structure thereto; and
a pressurizing part for applying a compressive force to the double-layer structure.

8. The device according to claim 7, further comprising an observing part for observing the double-layer structure.
